(19) United States
(12) Patent Application Publication
(10) Pub. No.: US 2026/0294368 A1
(43) Pub. Date: Oct. 1, 2026

(54) MOBILE RADIOGRAPHIC APPARATUS, NOTIFICATION METHOD, AND PROGRAM

(71) Applicant: KONICA MINOLTA, INC., Tokyo (JP)

(72) Inventor: Mao EGUCHI, Kokubunji-shi, Tokyo (JP)

(21) Appl. No.: 19/478,131

(22) PCT Filed: Mar. 22, 2024
(86) PCT No.: PCT/JP2024/011282

§ 371 Date: Oct. 23, 2025

(30) Foreign Application Priority Data
Apr. 28, 2023 (JP) 2023-074492

(51) Int. Cl. A61B 6/00; A61B 6/46

(52) U.S. Cl. CPC A61B 6/4405; A61B 6/461; A61B 6/54; A61B 6/56

(57) ABSTRACT

A mobile radiographic imaging apparatus includes: a plurality of housing sections each capable of housing a radiation detector that detects radiation; and a hardware processor that: acquires information on a radiation detector housed in one or more of the plurality of housing sections; and causes a notifier to make a notification about information indicating the one or more of the plurality of housing sections and the information on the radiation detector housed in the one or more of the plurality of housing sections.

### TECHNICAL FIELD

The present invention relates to a mobile radiographic imaging apparatus, a notification method, and a program.

### BACKGROUND ART

Conventionally, radiographic imaging has been performed using a movable body called a medical cart, in which a radiation generator is incorporated, and a portable radiographic imaging apparatus (radiation detector) wirelessly connected to the medical cart. In the radiographic imaging using a wireless connection, the radiation generator and the radiographic imaging apparatus need to be synchronized with each other.

Patent Document 1 describes a medical cart including a plurality of housing sections for housing portable radiographic imaging apparatuses. When a portable radiographic imaging apparatus is housed in the housing section, the radiographic imaging apparatus can be charged. That is, this housing section is a housing section capable of performing charging.

Patent Document 2 describes radiographic imaging by wirelessly connecting a radiation generator to a radiographic imaging apparatus. Patent Document 2 describes that, when a portable radiographic imaging apparatus is housed in a housing section, synchronization between the radiation generator and the radiographic imaging apparatus is performed simultaneously with charging. That is, this housing section is a housing section capable of performing charging and synchronization.

### CITATION LIST

### Patent Literature

Patent Document 1: JP 2015-100377A
Patent Document 2: JP 2006-305106A

### SUMMARY OF INVENTION

### Technical Problem

However, when the medical cart includes a plurality of housing sections, not all the housing sections are necessarily capable of performing synchronization. Specifically, some housing sections may be capable of performing charging and synchronization, while other housing sections may be capable of performing charging only. Therefore, there is a problem that a user does not know which housing section to use.

Furthermore, when the medical cart includes a plurality of housing sections capable of performing synchronization, there is a problem that the user does not know which of the radiographic imaging apparatuses housed in the plurality of housing sections capable of performing synchronization has completed synchronization.

Therefore, an object of the present invention is to make it easier for a user to select an appropriate housing section in a medical cart that includes a plurality of housing sections.

### Solution to Problem

In order to solve the above-described problem, a mobile radiographic imaging apparatus according to the present invention comprises: a plurality of housing sections each capable of housing a radiation detector that detects radiation; an acquisition section that acquires information on a radiation detector housed in one or more of the plurality of housing sections; and a controller that causes a notifier to make a notification about information indicating the one or more of the plurality of housing sections and the information on the radiation detector housed in the one or more of the plurality of housing sections.

A mobile radiographic imaging apparatus according to the present invention comprises: a plurality of housing sections each capable of housing a radiation detector that detects radiation; an acquisition section that acquires information on imaging; and a controller that, based on the information on the imaging acquired by the acquisition section, causes a notifier to make a notification about information indicating one or more of the plurality of housing sections and information on a radiation detector to be housed in the one or more of the plurality of housing sections.

A notification method according to the present invention is a notification method that uses a mobile radiographic imaging apparatus including a plurality of housing sections each capable of housing a radiation detector that detects radiation, the notification method comprising the steps of: acquiring information on a radiation detector housed in one or more of the plurality of housing sections; and making a notification, by a notifier, about information indicating the one or more of the plurality of housing sections and the information on the radiation detector housed in the one or more of the plurality of housing sections.

A notification method according to the present invention is a notification method that uses a mobile radiographic imaging apparatus including: a plurality of housing sections each capable of housing a radiation detector that detects radiation; and an acquisition section that acquires information on imaging, the notification method comprising the steps of: acquiring the information on the imaging; and, based on the information on the imaging acquired by the acquisition section, making a notification, by a notifier, about information indicating one or more of the plurality of housing sections and information on a radiation detector to be housed in the one or more of the plurality of housing sections.

A program according to the present invention causes a computer of a mobile radiographic imaging apparatus including a plurality of housing sections each capable of housing a radiation detector that detects radiation to function as: an acquisition section that acquires information on a radiation detector housed in one or more of the plurality of housing sections; and a controller that causes a notifier to make a notification about information indicating the one or more of the plurality of housing sections and the information on the radiation detector housed in the one or more of the plurality of housing sections.

A program according to the present invention causes a computer of a mobile radiographic imaging apparatus including: a plurality of housing sections each capable of housing a radiation detector that detects radiation; an acquisition section that acquires information on imaging to function as: an acquisition section that acquires the information on the imaging; and a controller that, based on the information on the imaging acquired by the acquisition section, causes a notifier to make a notification about information indicating one or more of the plurality of housing sections and information on a radiation detector to be housed in the one or more of the plurality of housing sections.

### Advantageous Effects of Invention

According to the present invention, in a medical cart that includes a plurality of housing sections, a user can easily select an appropriate housing section.

### BRIEF DESCRIPTION OF DRAWINGS

FIG. 1 This is a block diagram illustrating a radiographic imaging system according to an embodiment of the present invention.

FIG. 2 This is a schematic diagram illustrating an example of a configuration of a medical cart and a radiation detector.

FIG. 3 This is a block diagram illustrating a console.

FIG. 4 This is a flowchart illustrating a first housing section notification process executed by the console.

FIG. 5 This is a diagram illustrating an examination selection screen.

FIG. 6A This is a diagram illustrating information acquired for an FPD.

FIG. 6B This is a diagram illustrating the information acquired for an FPD.

FIG. 7 This is a diagram illustrating a pop-up notification.

FIG. 8 This is a diagram illustrating a pop-up notification.

FIG. 9 This is a flowchart illustrating a second housing section notification process executed by the console.

### DESCRIPTION OF EMBODIMENTS

Hereinafter, a radiographic imaging system according to an embodiment of the present invention will be described with reference to the drawings. However, the scope of the present invention is not limited to the following embodiment and drawings.

### Configuration

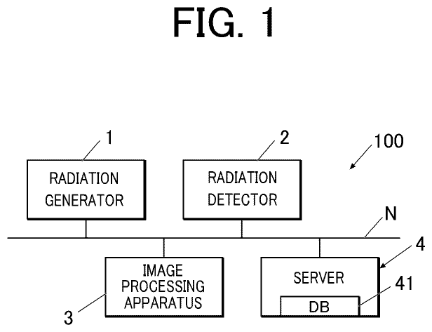

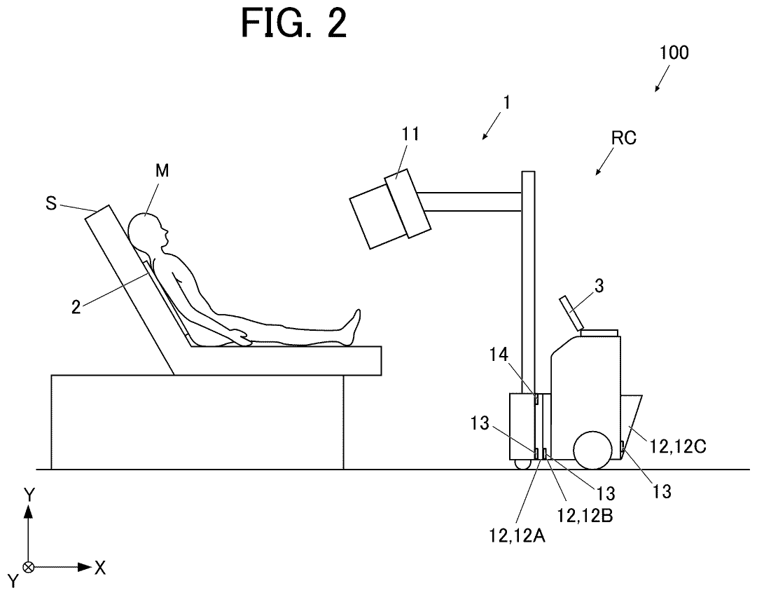

First, a schematic configuration of a radiographic imaging system 100 according to the present embodiment will be described with reference to FIGS. 1 and 2. FIG. 1 is a block diagram illustrating the radiographic imaging system 100. FIG. 2 is a schematic diagram illustrating an example of a configuration of a medical cart RC and a radiation detector 2.

As illustrated in FIG. 1, the radiographic imaging system 100 according to the present embodiment includes a radiation generator 1, the radiation detector 2, a console 3, and a server 4.

These are communicable with each other via a communication network N.

As illustrated in FIG. 2, the radiation generator 1 is incorporated in a mobile radiographic imaging apparatus called the medical cart RC together with the console 3 and the like.

Note that the radiographic imaging system 100 (X-ray imaging system 100) may be connectable to a hospital information system (HIS), a radiology information system (RIS), a picture archiving and communication system (PACS), an image analysis apparatus, and/or the like, which are not illustrated.

The radiation generator 1 includes a generator that applies a voltage corresponding to preset irradiation conditions (a tube voltage, a tube current, an irradiation time, a tube current-time product (mAs value), and the like), a radiation source 11 that generates radiation (for example, X-rays) of a dose corresponding to a tube current when a voltage is applied from the generator, and the like.

The radiation source 11 (tube) generates radiation (for example, X-rays) in a mode corresponding to a radiographic image to be captured. As illustrated in FIG. 2, the radiation source 11 is supported by an arm or the like included in the medical cart RC.

A housing section 12 houses the radiation detector 2, which will be described later. Specifically, the housing section 12 is a slot. Typical slot sizes are those suitable for radiation detectors 2 of 14×17 inches, 17×17 inches, and 10×12 inches. The medical cart RC includes a plurality of housing sections 12. Each of the plurality of housing sections 12 includes a power supplier 13 and/or a synchronization signal output section (synchronization signal outputter) 14. The function of the housing section 12 with respect to the radiation detector refers to the function of the power supplier 13 and/or the function of the synchronization signal output section 14, among others. The medical cart RC in the example of FIG. 2 includes one housing section 12A including the power supplier 13 and the synchronization signal output section 14, and two housing sections 12B and 12C each including only the power supplier 13. Furthermore, in the medical cart RC in the example of FIG. 2, for example, the slot size of the housing section 12A is 17×17 inches, the slot size of the housing section 12B is 14×17 inches, and the slot size of the housing section 12C is 10×12 inches.

The housing section 12 includes a connector (not illustrated) that communicatively connects the radiation detector 2 and the console 3. When the radiation detector 2 is housed, the housing section 12 transmits information on the radiation detector 2 to the console 3, which will be described later, via the connector. When the radiation detector 2 to be described later is housed in the housing section 12, the power supplier 13 supplies electric power to the radiation detector 2. The power supplier 13 supplies electric power to the radiation detector 2, acquires information on the remaining battery level of the radiation detector 2, and transmits the information to the console 3 to be described later.

The synchronization signal output section 14 outputs a synchronization signal for synchronizing the radiation generator 1 with the radiation detector 2 under the control of a controller 31 to be described later. In wireless serial imaging (wireless moving image capture), the radiation generator 1 and the radiation detector 2 need to be synchronized with each other.

The synchronization signal will be described. A synchronization signal for synchronizing the timing at which radiation is emitted from the radiation generator 1 with the timing at which a radiographic image is generated is generated by the synchronization signal output section 14. The generated synchronization signal is transmitted to the radiation detector 2 when the radiation detector 2 is housed in the housing section 12.

Note that the synchronization signal may be automatically transmitted to the radiation detector 2 when the radiation detector 2 is housed in the housing section 12. The synchronization signal may be transmitted to the radiation detector 2 based on a control signal from an operation part 35, which will be described later, by a user.

Although not illustrated, the radiation detector 2 (flat panel detector (FPD)) includes a circuit board, a scanning circuit, a readout circuit, a controller, an output section, and the like. On the circuit board, radiation detection elements that generate electric charges corresponding to doses upon receiving radiation and pixels including switching elements that accumulate and release the electric charges are two-dimensionally aligned (in a matrix). The scanning circuit turns on and off each switching element. The readout circuit reads out the amounts of electric charges released from the respective pixels as signal values. The controller generates a radiographic image from the signal values read out by the readout circuit. The output section outputs data of the generated radiographic image and the like to the outside.

When radiation is emitted from the radiation generator 1 during radiographic image capture, the radiation detector 2 generates a radiographic image based on the synchronization signal.

The radiation detector 2 may store information on the radiation detector to be described later.

Typical sizes of the radiation detector 2 are 14×17 inches, 17×17 inches, and 10×12 inches.

The radiation detector 2 is portable (cassette type). During the radiographic image capture, as illustrated in FIG. 2, the radiation detector 2 is loaded on an imaging table S.

After the radiation generator 1 and the radiation detector 2 are synchronized with each other, a deviation may occur in synchronization over time. Therefore, a synchronization maintaining time is set for the radiation detector 2. During the synchronization maintaining time, the radiation generator 1 and the radiation detector 2 are assumed to be synchronized with each other.

Note that the radiation detector 2 may be a detector that incorporates a scintillator or the like, converts emitted radiation into light of another wavelength such as visible light by the scintillator, and generates charges corresponding to the converted light (so-called indirect type) or may be a detector that generates charges directly from radiation without a scintillator or the like (so-called direct type).

The console 3 constitutes an image processing apparatus and an electronic device and includes a PC, a dedicated device, and the like.

The console 3 can synchronize the radiation generator 1 with the radiation detector 2 by transmitting a synchronization signal to the radiation generator 1. The console 3 can synchronize the radiation generator 1 with the radiation detector 2 by causing the synchronization signal output section 14 to transmit a synchronization signal to the radiation detector 2.

The console 3 can set various imaging conditions (a tube voltage, a tube current, an irradiation time, a tube current-time product (mAs value), a frame rate, a body size of a subject H, the presence or absence of a grid, and the like) in an imaging apparatus and the like based on imaging order information acquired from another system (HIS, RIS, or the like) or an operation by the user.

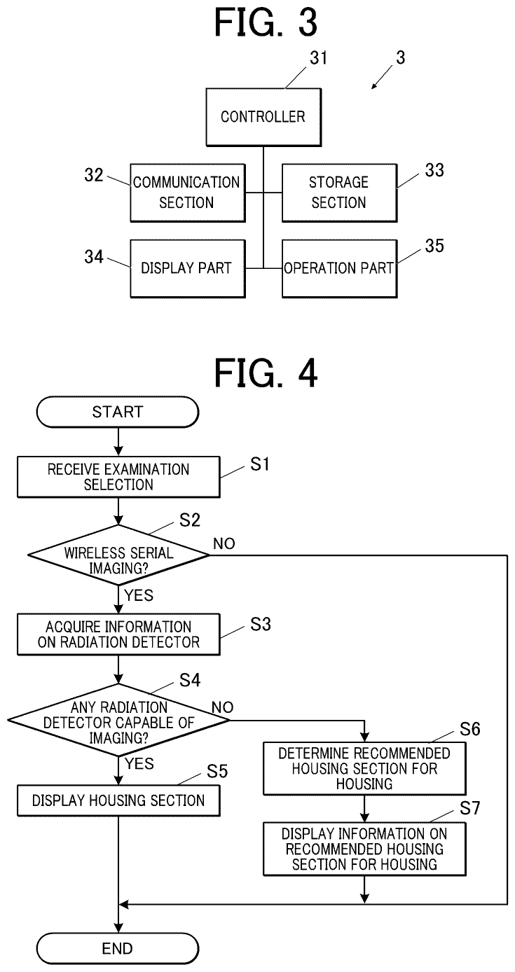

As illustrated in FIG. 3, the console 3 according to the present embodiment includes a controller 31, a communication section 32, a storage section 33, a display part 34, and an operation part 35.

The components 31 to 35 are electrically connected to each other by a bus.

The controller 31 includes a central processing unit (CPU), a random-access memory (RAM), and the like.

The CPU of the controller 31 reads various programs stored in the storage section 33, develops the programs in the RAM, executes various processes in accordance with the developed programs, and centrally controls the operation of each component of the console 3.

The controller 31 has a function of executing a first housing section notification process illustrated in FIG. 4, a second housing section notification process illustrated in FIG. 9, and the like.

The controller 31 functions as an acquisition section that acquires information on a radiation detector 2 housed in a housing section 12.

The controller 31 functions as a controller that causes a notifier (such as the display part 34) to make a notification about information indicating a housing section 12 and information on the radiation detector 2 housed in the housing section 12.

The controller 31 functions as an acquisition section that acquires information on imaging.

The controller 31 functions as a controller that causes a notifier (such as the display part 34) to make a notification about information indicating a housing section 12 and information on the radiation detector 2 to be housed in the housing section 12 based on the information on imaging acquired by the acquisition section (controller 31).

The communication section 32 includes a communication module and the like.

The communication section 32 transmits and receives various signals and various data to and from other devices or the like connected via the communication network N (a local area network (LAN), a wide area network (WAN), the Internet, or the like).

The storage section 33 includes a nonvolatile semiconductor memory, a hard disk, or the like.

The storage section 33 stores various programs to be executed by the controller 31, parameters necessary for executing the programs, and the like.

The display part 34 includes a display device that displays an image, such as a liquid crystal display (LCD) or a cathode ray tube (CRT), a lamp (such as an LED) that emits light, a speaker that outputs sound, a transducer that vibrates, and the like.

The display part 34 functions as a notifier that makes a notification based on control from the controller 31.

The operation part 35 includes a keyboard having cursor keys, number input keys, various function keys, and the like, a pointing device such as a mouse, a touch screen layered on a surface of the display device, and the like.

The operation part 35 outputs a control signal corresponding to an operation performed by the user to the controller 31.

The server 4 includes a PC, a dedicated device, a virtual server on the cloud, or the like.

Further, the server 4 includes a database 41.

The database 41 stores the information on the radiation detectors and the information on imaging.

The information on the radiation detectors is managed based on an individual identification number for identifying each radiation detector 2. The information on the radiation detectors includes, for example, panel size, wireless serial imaging capability, synchronization state, remaining battery level, and housing section. The synchronization state indicates whether the radiation generator 1 and the radiation detector 2 are synchronized with each other. The housing section indicates which housing section of the medical cart RC houses the radiation detector 2.

The information on imaging is managed based on an imaging ID for identifying each imaging. The information on imaging includes patient information and imaging information. The patient information includes, for example, weight, height, and body thickness. The imaging information includes, for example, an imaging method such as wireless serial imaging, an SID, a tube voltage, a slit width, a filter, and a required panel size.

Note that although the database 41 is provided in the server 4 independent of the console 3 and the like in the present embodiment, the database 41 may be provided in the console 3 or another apparatus included in the radiographic imaging system 100.

When another system such as a PACS is connected to the radiographic imaging system 100, the database 41 may be provided in the other system.

In the radiographic imaging system 100 according to the present embodiment configured as described above, the radiation source of the radiation generator 1 and the radiation detector 2 are wirelessly connected to each other and disposed to face each other with a gap between the radiation source and the radiation detector 2, and radiation is emitted from the radiation source to the subject H located between the radiation source and the radiation detector 2. This allows a radiographic image of the subject H to be captured. When a radiographic image is a moving image (serial imaging), emission of pulsed radiation and generation of a frame image are repeated a plurality of times in a short time (e.g., fifteen times per second) for each imaging operation.

### First Housing Section Notification Process

Next, the first housing section notification process will be described with reference to a flowchart of FIG. 4. The first housing section notification process is a process in which, based on the information on a radiation detector 2 housed in a housing section 12, the controller 31 causes the notifier to make a notification about the information indicating the housing section 12 that houses the radiation detector 2 and information indicating the state of the radiation detector 2.

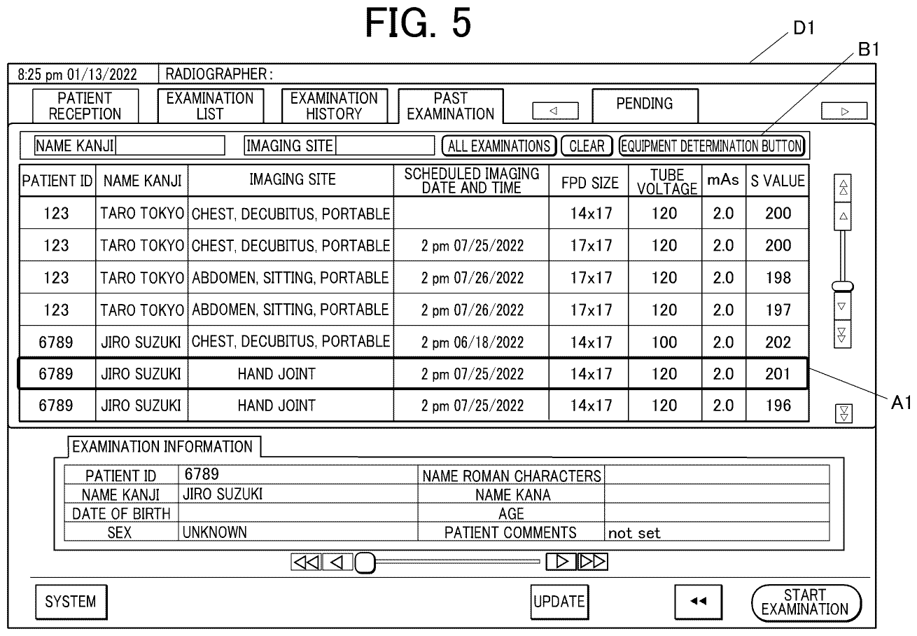

First, the user selects an examination using the operation part 35 on an examination selection screen illustrated in FIG. 5 displayed on the display part 34 and presses an equipment determination button B1. The controller 31 receives the selected examination (step S1).

Here, the examination selection screen D1 illustrated in FIG. 5 will be described.

A region A1 is a region for displaying a list of examinations.

The equipment determination button B1 is a button for determining whether various apparatuses of the radiographic imaging system 100 are compatible with the examination selected in the region A1. In the flowchart of FIG. 4, the equipment determination button B1 functions as a button for determining whether various apparatuses are compatible with wireless serial imaging.

Next, the controller 31 determines whether the examination received in step S1 is wireless serial imaging (step S2). If the examination is wireless serial imaging (step S2; YES), the first housing section notification process proceeds to step S3. If the examination is not wireless serial imaging (step S2; YES), the first housing section notification process ends.

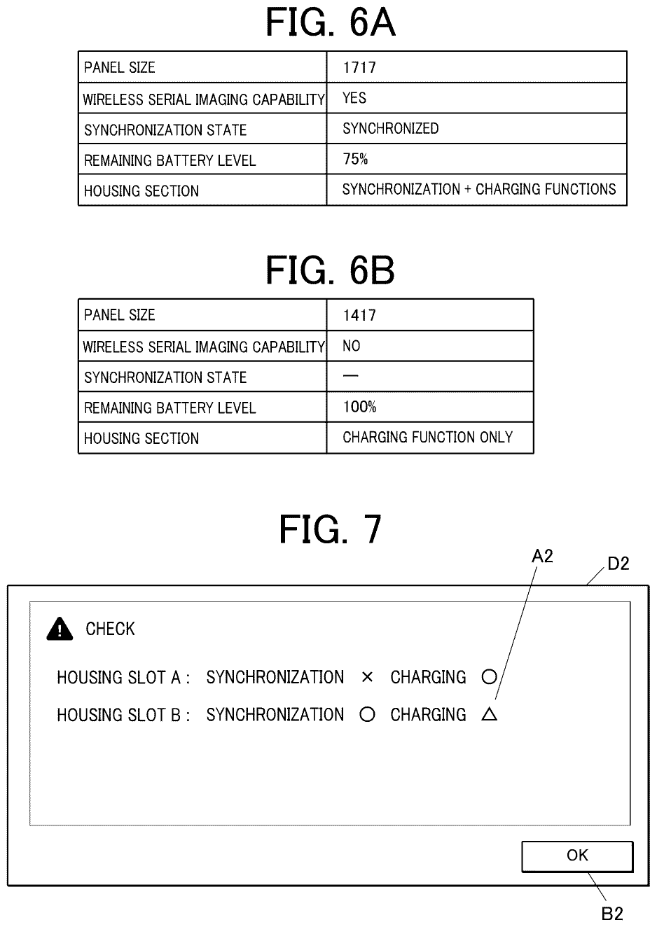

Next, the controller 31 acquires, for example, the information on a radiation detector 2 as illustrated in FIG. 6A and FIG. 6B for each housing section 12 that houses the radiation detector 2 (step S3; acquisition step).

Here, the acquisition of the information on a radiation detector 2 illustrated in FIG. 6 will be described. When a radiation detector 2 is housed in a housing section 12, the controller 31 may acquire the information on the radiation detector 2 from the radiation detector 2 itself. The controller 31 may acquire the individual identification number for identifying the radiation detector 2 from the radiation detector 2 and acquire the information on the radiation detector 2 from the storage section 33 or the database 41 based on the individual identification number.

For example, among the information on the radiation detector 2, the controller 31 acquires the synchronization state and the remaining battery level from the radiation detector 2. When the radiation detector 2 is housed in the housing section 12 and connected to the console 3, the controller 31 acquires the information from the radiation detector 2. In addition, the controller 31 may acquire information on the panel size or wireless serial imaging capability of the radiation detector 2 from the radiation detector 2 in the same manner as described above or from the storage section 33 or the database 41 based on the individual identification number.

Next, the controller 31 determines whether there is a radiation detector 2 capable of performing wireless serial imaging based on the information on the radiation detector 2 (step S4). If there is (step S4; YES), the first housing section notification process proceeds to step S5. If not (step S4; NO), the first housing section notification process proceeds to step S6. That there is a radiation detector 2 capable of performing wireless serial imaging means that a radiation detector 2 capable of performing wireless serial imaging is housed in a housing section 12.

Next, if there is a radiation detector 2 capable of performing wireless serial imaging (step S4; YES), the controller 31 causes the display part 34 to display information including the housing section that houses the radiation detector 2 capable of performing wireless serial imaging based on the information on the radiation detector 2 (step S5; notification step). Specifically, the controller 31 causes the display part 34 to display, for example, a pop-up notification D2 illustrated in FIG. 7.

Step S4 is YES even when the radiation detector 2 capable of performing wireless serial imaging housed in the housing section 12 is not synchronized. The user can check the synchronization status by checking the pop-up notification D2. If not synchronized, the user synchronizes the radiation generator 1 with the radiation detector 2.

After completion of the operation in step S5, the first housing section notification process ends.

Here, the pop-up notification D2 illustrated in FIG. 7 will be described.

A region A2 is a region for displaying information indicating a housing section 12 of the medical cart RC that houses a radiation detector 2 and information indicating the state of the radiation detector 2. In other words, information on a radiation detector 2 that the user can use immediately afterwards is displayed in the region A2. The state of the radiation detector 2 is information on synchronization and charging of the radiation detector 2. In the example of FIG. 7, it is shown that the radiation detector 2 housed in the housing slot A (housing section) is asynchronous and fully charged. On the other hand, it is shown that the radiation detector 2 housed in the housing slot B (housing section) has completed synchronization and is being charged.

The controller 31 may display, in the region A2, only information indicating a housing section 12 that houses a radiation detector 2 that has completed synchronization and information indicating the state of the radiation detector 2 that has completed synchronization. The controller 31 may display, in the region A2, only the information indicating a housing section 12 that houses a radiation detector 2 that has completed synchronization.

The controller 31 may display, in the region A2, only information indicating a housing section 12 that houses a radiation detector 2 that has been charged by the power supplier 13 to a predetermined value or more and information indicating the state of the radiation detector 2 that has been charged by the power supplier 13 to a predetermined value or more. The controller 31 may display, in the region A2, only the information indicating a housing section 12 that houses a radiation detector 2 that has been charged by the power supplier 13 to a predetermined value or more.

An OK button B2 is a button to be pressed when the user checks the pop-up notification D2. When the OK button B2 is pressed, the pop-up notification D2 is closed.

Next, when there is no radiation detector 2 capable of performing wireless serial imaging (step S4; NO), the controller 31 determines a housing section that is to house a radiation detector 2 capable of wireless serial imaging (step S6).

The controller 31 determines a housing section 12 that includes the synchronization signal output section 14 as the housing section that is to house a radiation detector 2 capable of performing wireless serial imaging.

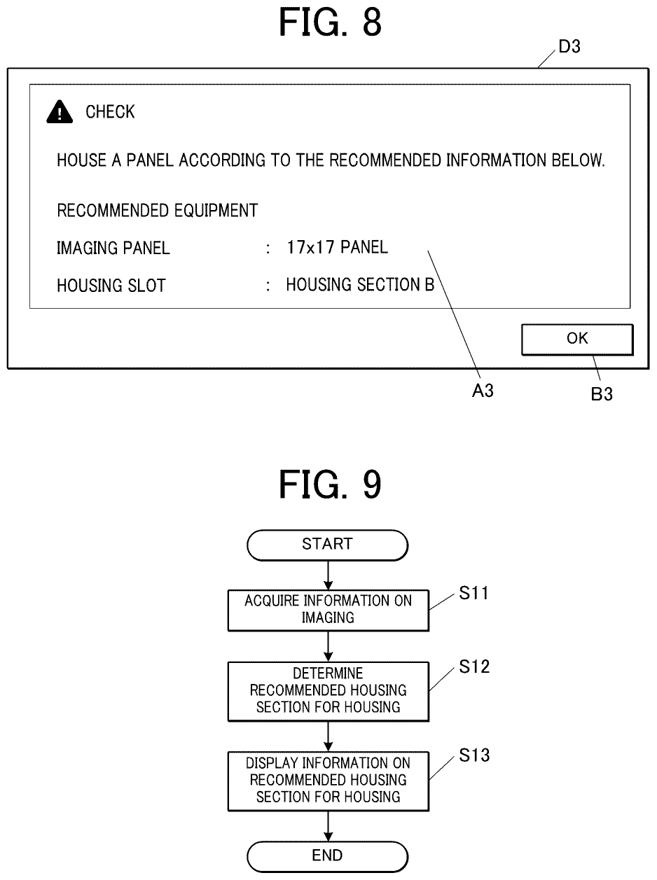

Next, the controller 31 causes the display part 34 to display the housing section that is to house a radiation detector 2 capable of performing wireless serial imaging (step S7). Specifically, the controller 31 causes the display part 34 to display, for example, a pop-up notification D3 illustrated in FIG. 8.

After completion of the operation in step S7, the first housing section notification process ends.

Here, the pop-up notification D3 illustrated in FIG. 8 will be described.

A region A3 is a region for displaying information on a housing section of the medical cart RC that is to house a radiation detector 2. In the example of FIG. 8, it is shown that the housing section B is to house a panel having a size of 17×17.

An OK button B3 is a button to be pressed when the user checks the pop-up notification D3. When the OK button B3 is pressed, the pop-up notification D3 is closed.

### Others

Various configuration examples other than the above description will be described. After the radiation generator 1 and the radiation detector 2 are synchronized with each other, a deviation may occur in synchronization over time. In particular, when the remaining battery level of the radiation detector 2 is low, a deviation is likely to occur in synchronization. Therefore, it is desirable that the controller 31 determine the synchronization maintaining time in accordance with the acquired information on the remaining battery level. When the synchronization maintaining time has elapsed, the controller 31 may cause the synchronization signal to be output again.

The controller 31 may turn off the function of the power supplier 13 when the remaining battery level is full in the acquired information on the remaining battery level of the radiation detector 2.

The controller 31 may be able to set the timing at which the radiation generator 1 and the radiation detector 2 are synchronized with each other. This results in synchronization not always being up to date. However, by having the controller 31 output the synchronization signal at predetermined intervals, such as every 20 minutes, power-saving effects are achieved.

In step S5, the controller 31 causes the display part 34 to display the housing section to house. However, the controller 31 may allow the user to select a housing section 12 that houses a radiation detector 2 that the user wants to use to cause the synchronization signal output section 14 of that housing section 12 to operate, thereby synchronizing the radiation generator 1 with the radiation detector 2. In this case, as a prerequisite, each housing section 12 needs to include the synchronization signal output section 14.

### Second Housing Section Notification Process

Next, the second housing section notification process will be described with reference to a flowchart of FIG. 9. The second housing section notification process is a process in which the controller 31 causes the notifier to make a notification about information indicating a housing section that is to house a radiation detector based on the acquired information on imaging.

First, the controller 31 acquires the information on imaging (step S11; acquisition step).

The information on imaging includes at least one of information on the user who uses the medical cart RC, examination information, the size of the radiation detector 2 to be used for imaging, or information set in the medical cart RC. The information on the user who uses the medical cart RC is input to the controller 31 by the user using the operation part 35.

Next, the controller 31 determines a housing section that is to house a radiation detector 2 (step S12). In the second housing section notification process, a recommended housing section is determined based on the information on imaging, without being limited to a radiation detector 2 capable of performing wireless serial imaging.

For example, when wireless serial imaging is included in the acquired examination information, the controller 31 determines a housing section 12 capable of performing synchronization as the recommended housing section.

For example, when the maximum size of the radiation detector 2 used for imaging included in the acquired examination information is the 14×17 size, the controller 31 determines a housing section 12 suitable for housing a radiation detector 2 of the 14×17 size as the recommended housing section.

Next, as in the first housing section notification process, the controller 31 causes the display part 34 to display the pop-up notification D3 illustrated in FIG. 8 (step S13; notification step).

### Others

In the above example, the controller 31 determines in step S12 the recommended housing section based on the examination information, but the present invention is not limited thereto.

For example, the controller 31 may determine the recommended housing section based on the information on the user such as the height of the user who uses the medical cart RC. That is, the controller 31 determines, as the recommended housing section, a housing section 12 located at a lower position when the user is short and a housing section 12 located at a higher position when the user is tall.

Furthermore, for example, the controller 31 may determine the recommended housing section based on the information set in the medical cart RC. The information set in the medical cart RC is set in advance according to the placement and use of the medical cart, such as an emergency, a hospital ward, and an operation.

Furthermore, for example, the controller 31 may determine the recommended housing section based on the information on the user who has logged in to the console 3 of the medical cart RC. That is, the recommended housing section may be set for each user.

In addition, for example, the controller 31 may determine the recommended housing section according to an imaging condition. For example, when the necessary panel size is the 14×17 size in the imaging condition, the controller 31 may determine a housing section 12 that can house the radiation detector 2 having a size equal to or larger than the 14×17 size as the recommended housing section.

The number of recommended housing sections is not limited to one and may be plural. When there is a plurality of recommended housing sections, a recommended housing section that is preferentially recommended may be set in advance.

When the size of the housing section 12 is variable under the control of the controller 31, the controller 31 may change the size of the housing section 12 determined as the recommended housing section. For example, when the size of the housing section 12 in the thickness direction is variable, by increasing the size of the housing section 12 in the thickness direction, which is determined as the recommended housing section, the user can easily insert and remove the radiation detector 2.

The controller 31 preferentially determines the housing section 12 having the largest size in the thickness direction as the recommended housing section, so that the user can easily insert and remove the radiation detector 2.

When there is a plurality of housing sections 12 on the near side and the far side as in the medical cart RC of FIG. 2, the controller 31 may preferentially determine the housing section on the near side as the recommended storage section. Accordingly, the radiation detector 2 can be easily inserted and removed. Basically, since the radiation source 11 (tube) is disposed on the far side, the user finds the housing section 12 on the near side easier to use. Depending on the movement of the radiation source 11, the housing section 12 on the far side may be easier to use in some cases. In this case, the controller 31 may preferentially determine the housing section on the far side as the recommended housing section.

When there is a plurality of housing sections 12 on the near side of the medical cart RC, the controller 31 may determine the housing section 12 on the nearest side as the recommended housing section.

The housing section 12 preferentially determined as the recommended housing section by the controller 31, as described above, can be set by the user.

### Other Modification Examples

In the above description, the display part 34 functions as the notifier. However, the notifier may be a speaker or the like that emits sound. In addition, the notifier may be a light-emitting part such as a lamp disposed in the housing section 12 or in the vicinity of the housing section 12. The vicinity refers to a position at which the user can visually recognize the housing section 12 and the notifier at the same time. Furthermore, the notifier may be a light-emitting part such as a lamp disposed in the radiation detector 2.

A radiation detector 2 is housed, and the controller 31 acquires the information on the radiation detector 2 acquired by the housing section 12. According to the information on a radiation detector 2 acquired by the controller 31, it is determined that the radiation detector 2 is a radiation detector that cannot be synchronized. In this case, the synchronization signal output section 14 (controller 31) may be configured not to automatically synchronize the radiation generator 1 with the radiation detector 2.

When the medical cart RC is used in a facility where only still image capture is performed, the synchronization function of the radiation detector 2 may be turned off. This provides a battery-saving effect for the radiation detector 2.

The synchronization maintaining time of the radiation detector 2 may be set to a suitable value for each facility. This provides a battery-saving effect for the radiation detector 2.

When the radiation detector 2 is synchronized with the radiation generator 1, the controller 31 may indicate the synchronization on the display part 34. In addition, the controller 31 may indicate the synchronization by turning on a lamp or outputting sound from a speaker.

Regarding the housing section 12, there may be a plurality of housing sections 12 or a single housing section 12 capable of performing charging and synchronization. Having a single housing section 12 capable of performing charging and synchronization can reduce costs and power consumption. When there is a plurality of housing sections 12 capable of performing charging and synchronization, a plurality of radiation detectors 2 can be synchronized, and thus the number of radiation detectors 2 that can perform imaging can be increased.

In a case where there is a plurality of housing sections 12 capable of performing synchronization, when synchronization is started in one housing section 12, synchronization may not be performed in the other housing sections 12, or synchronization may be simultaneously performed in all the housing sections 12.

Depending on the situation in which the user uses the medical cart RC, the housing section 12 that is easy for the user to use may be set as the recommended housing section by the controller 31.

Functions of the housing section 12, such as synchronization, charging, and synchronization +charging, may be settable by the user. That is, even a housing section 12 capable of performing charging and synchronization can have only the charging function activated by the user. The function may be changed by pressing a switch disposed in the housing section 12 or in the vicinity of the housing section 12 or may be set using the operation part 35 of the console 3. The vicinity refers to a position where the user can visually recognize the housing section 12 and the switch at the same time.

The medical cart RC may include housing sections 12 having different charging capabilities. The controller 31 may make a notification about a housing section 12 having a charging capability corresponding to the remaining battery level of a radiation detector 2 as the recommended housing section. Specifically, for example, when the remaining battery level of a radiation detector 2 is extremely low, the controller 31 determines a housing section 12 having a high charging capability as the recommended housing section.

### Effects and the Like

In view of the above, a mobile radiographic imaging apparatus (medical cart RC) includes: a plurality of housing sections 12 each capable of housing a radiation detector 2 that detects radiation; an acquisition section (controller 31) that acquires information on a radiation detector 2 housed in one of the plurality of housing sections 12; and the controller 31 that causes a notifier (display part 34 or the like) to make a notification about information indicating the one of the plurality of housing sections 12 and the information on the radiation detector 2 housed in the one of the plurality of housing sections 12. This makes it easier for the user to select an appropriate housing section in the medical cart having the plurality of housing sections.

A mobile radiographic imaging apparatus (medical cart RC) includes: a plurality of housing sections 12 each capable of housing a radiation detector 2 that detects radiation; an acquisition section (controller 31) that acquires information on imaging; and the controller 31 that causes a notifier (display part 34 or the like) to make a notification about information indicating one of the plurality of housing sections 12 and information on a radiation detector 2 to be housed in the one of the plurality of housing sections 12 based on the information on the imaging acquired by the acquisition section (controller 31). This makes it easier for the user to select an appropriate housing section in the medical cart having the plurality of housing sections.

A notification method uses a mobile radiographic imaging apparatus (medical cart RC) that includes a plurality of housing sections 12 each capable of housing a radiation detector 2 that detects radiation. The notification method includes the steps of: acquiring information on a radiation detector 2 housed in one of the plurality of housing sections 12 (step S3); and making a notification, by a notifier (display part 34 or the like), about information indicating the one of the plurality of housing sections 12 and the information on the radiation detector 2 housed in the one of the plurality of housing sections 12 (step S5). This makes it easier for the user to select an appropriate housing section in the medical cart having the plurality of housing sections.

A notification method uses a mobile radiographic imaging apparatus (medical cart RC) that includes: a plurality of housing sections 12 each capable of housing a radiation detector 2 that detects radiation; and an acquisition section (controller 31) that acquires information on imaging. The notification method includes the steps of: acquiring the information on the imaging (step S11); and, based on the information on the imaging acquired in the acquiring (step S11), making a notification, by a notifier (display part 34 or the like), about information indicating one of the plurality of housing sections 12 and information on a radiation detector 2 to be housed in the one of the plurality of housing sections 12 (step S13). This makes it easier for the user to select an appropriate housing section in the medical cart having the plurality of housing sections.

A program causes a computer of a mobile radiographic imaging apparatus (medical cart RC) that includes a plurality of housing sections 12 each capable of housing a radiation detector 2 that detects radiation to function as: an acquisition section (controller 31) that acquires information on a radiation detector 2 housed in one of the plurality of housing sections 12; and the controller 31 that causes a notifier (display part 34 or the like) to make a notification about information indicating the one of the plurality of housing sections 12 and the information on the radiation detector 2 housed in the one of the plurality of housing sections 12. This makes it easier for the user to select an appropriate housing section in the medical cart having the plurality of housing sections.

A program causes a computer of a mobile radiographic imaging apparatus (medical cart RC) that includes: a plurality of housing sections 12 each capable of housing a radiation detector 2 that detects radiation; and an acquisition section (controller 31) that acquires information on imaging to function as: an acquisition section (controller 31) that acquires the information on the imaging; and the controller 31 that causes a notifier (display part 34 or the like) to make a notification about information indicating one of the plurality of housing sections 12 and information on a radiation detector 2 to be housed in the one of the plurality of housing sections 12 based on the information on the imaging acquired by the acquisition section (controller 31). This makes it easier for the user to select an appropriate housing section in the medical cart having the plurality of housing sections.

Although the present invention has been described in detail based on the embodiment, the present invention is not limited to the above-described embodiment. The embodiment can be modified without departing from the spirit and scope of the invention.

For example, in the above-described embodiment, the console 3 has the function of executing the housing section notification processes. However, another apparatus included in the radiographic imaging system 100 or another system connected to the radiographic imaging system 100 may have the function of executing the housing section notification processes or a part of the function.

Further, in the above description, an example in which a semiconductor memory or a hard disk is used as a computer-readable medium of the program according to the present invention has been disclosed, but the present invention is not limited to this example.

As another computer-readable medium, a nonvolatile memory such as a flash memory or a portable recording medium such as a CD-ROM can be applied.

As a medium for providing data of the program according to the present invention via a communication line, a carrier wave is also applied to the present invention.

### INDUSTRIAL APPLICABILITY

The present disclosure can be used for a mobile radiographic imaging apparatus, a notification method, and a program.

### REFERENCE SIGNS LIST

100 radiographic imaging system
RC medical cart (mobile radiographic imaging apparatus)
1 radiation generator
11 radiation source
12 housing section
13 power supplier
14 synchronization signal output section
2 radiation detector
3 console
31 controller (acquisition section)
32 communication section
33 storage section
34 display part (notifier, operation screen)
35 operation part
4 server
41 database
N communication network

1. A mobile radiographic imaging apparatus comprising:
a plurality of housing sections each capable of housing a radiation detector that detects radiation; and
a hardware processor that:
acquires information on a radiation detector housed in one or more of the plurality of housing sections; and
causes a notifier to make a notification about information indicating the one or more of the plurality of housing sections and the information on the radiation detector housed in the one or more of the plurality of housing sections.

2. The mobile radiographic imaging apparatus according to claim 1, wherein the information on the radiation detector includes at least one of a size of the radiation detector, whether the radiation detector is capable of performing wireless moving image capture, or a remaining battery level of the radiation detector.

3. The mobile radiographic imaging apparatus according to claim 1, further comprising:
a radiation generator; and
a synchronization signal outputter that outputs a synchronization signal for performing wireless moving image capture between the radiation generator and the radiation detector, wherein
the hardware processor causes the notification to be made, the notification being about the information indicating the one or more of the plurality of housing sections that houses the radiation detector that has completed synchronization with the radiation generator by using the synchronization signal output by the synchronization signal outputter.

4. The mobile radiographic imaging apparatus according to claim 1, further comprising a power supplier that supplies electric power to the radiation detector, wherein the hardware processor causes the notification to be made, the notification being about the information indicating the one or more of the plurality of housing sections that houses the radiation detector that has been charged by the power supplier to a predetermined level or more.

5. The mobile radiographic imaging apparatus according to claim 1, wherein the plurality of housing sections differs from each other in at least one of a function for a radiation detector housed in each of the plurality of housing sections or a size of a radiation detector that each of the plurality of housing sections is capable of housing.

6. The mobile radiographic imaging apparatus according to claim 1, wherein the notifier is disposed in at least one of each of the plurality of housing sections, a vicinity of each of the plurality of housing sections, or an operation screen of the mobile radiographic imaging apparatus.

7. A mobile radiographic imaging apparatus comprising:
a plurality of housing sections each capable of housing a radiation detector that detects radiation; and
a hardware processor that: acquires information on imaging; and
based on the acquired information on the imaging, causes a notifier to make a notification about information indicating one or more of the plurality of housing sections and information on a radiation detector to be housed in the one or more of the plurality of housing sections.

8. The mobile radiographic imaging apparatus according to claim 7, wherein the information on the imaging includes at least one of information on a user of the mobile radiographic imaging apparatus, examination information, a size of a radiation detector to be used for the imaging, or information on the mobile radiographic imaging apparatus.

9. The mobile radiographic imaging apparatus according to claim 8, further comprising:
a radiation generator; and
a synchronization signal outputter that outputs a synchronization signal for performing wireless moving image capture between the radiation generator and a radiation detector, wherein
in response to acquiring an imaging order of the wireless moving image capture as the examination information, the hardware processor causes the notification to be made, the notification being about information indicating one or more of the plurality of housing sections capable of outputting the synchronization signal from the synchronization signal outputter to the radiation detector.

10. The mobile radiographic imaging apparatus according to claim 8, wherein, based on the acquired examination information, the hardware processor causes the notification to be made, the notification being about the one or more of the plurality of housing sections that is to house the radiation detector.

11. The mobile radiographic imaging apparatus according to claim 7, wherein the plurality of housing sections differs from each other in at least one of a function for a radiation detector housed in each of the plurality of housing sections or a size of a radiation detector that each of the plurality of housing sections is capable of housing.

12. The mobile radiographic imaging apparatus according to claim 7, wherein the notifier is disposed in at least one of each of the plurality of housing sections, a vicinity of each of the plurality of housing sections, or an operation screen of the mobile radiographic imaging apparatus.

13. A notification method that uses a mobile radiographic imaging apparatus including a plurality of housing sections each capable of housing a radiation detector that detects radiation, the notification method comprising:
acquiring information on a radiation detector housed in one or more of the plurality of housing sections; and
making a notification, by a notifier, about information indicating the one or more of the plurality of housing sections and the information on the radiation detector housed in the one or more of the plurality of housing sections.

14. A notification method that uses a mobile radiographic imaging apparatus including:
a plurality of housing sections each capable of housing a radiation detector that detects radiation; and
a hardware processor that acquires information on imaging;
the notification method comprising:
acquiring the information on the imaging; and,
based on the information on the imaging acquired in the acquiring, making a notification, by a notifier, about information indicating one or more of the plurality of housing sections and information on a radiation detector to be housed in the one or more of the plurality of housing sections.

15. A non-transitory computer-readable recording medium storing a computer-executable program, the program causing a computer of a mobile radiographic imaging apparatus including a plurality of housing sections each capable of housing a radiation detector that detects radiation to:
acquire information on a radiation detector housed in one or more of the plurality of housing sections; and
cause a notifier to make a notification about information indicating the one or more of the plurality of housing sections and the information on the radiation detector housed in the one or more of the plurality of housing sections.

16. A non-transitory computer-readable recording medium storing a computer-executable program, the program causing a computer of a mobile radiographic imaging apparatus including:
a plurality of housing sections each capable of housing a radiation detector that detects radiation; and
a hardware processor that acquires information on imaging to:
acquire the information on the imaging; and
based on the acquired information on the imaging, cause a notifier to make a notification about information indicating one or more of the plurality of housing sections and information on a radiation detector to be housed in the one or more of the plurality of housing sections.
